(19) United States
(12) Patent Application Publication
(10) Pub. No.: US 2015/0139518 A1
(43) Pub. Date: May 21, 2015

(54) IMAGE PROCESSING APPARATUS

(71) Applicants: Kabushiki Kaisha Toshiba, Minato-ku (JP); Toshiba Medical Systems Corporation, Otawara-shi (JP)

(72) Inventors: Shumpei OOHASHI, Otawara (JP); Rie OCHIAI, Nasushiobara (JP); Haruki IWAI, Otawara (JP); Shingo ABE, Nasushiobara (JP); Takayuki TOMISAKI, Nasushiobara (JP)

(73) Assignees: Kabushiki Kaisha Toshiba, Minato-ku (JP); Toshiba Medical Systems Corporation, Otawara-shi (JP)

(21) Appl. No.: 14/570,860

(22) Filed: Dec. 15, 2014

Related U.S. Application Data
(63) Continuation of application No. PCT/JP2013/068738, filed on Jul. 9, 2013.

(30) Foreign Application Priority Data
Jul. 9, 2012 (JP) 2012-153623

(51) Int. Cl. A61B 6/00; G06T 7/00; A61B 8/08

(52) U.S. Cl. CPC A61B 6/463; A61B 6/502; A61B 6/469; A61B 8/0825; G06T 7/0022; G06T 7/0016; G06T 2207/10132; G06T 2207/20104; G06T 2207/30068; G06T 2210/41
USPC 382/131

(57) ABSTRACT

An image processing apparatus according to an embodiment includes an input unit, an identifying unit, and a display control unit. The input unit receives specification of a region of interest (ROI) included in a mammography image. The identifying unit identifies a medical image including a position substantially the same as the position of the ROI received by the input unit in an ultrasound image group acquired from a subject for whom the mammography image is captured. The display control unit performs control such that the medical image identified by the identifying unit is displayed on a display unit.

### CROSS-REFERENCE TO RELATED APPLICATIONS

This application is a continuation of PCT international application Ser. No. PCT/JP2013/068738 filed on Jul. 9, 2013 which designates the United States, incorporated herein by reference, and which claims the benefit of priority from Japanese Patent Application No. 2012-153623, filed on Jul. 9, 2012, the entire contents of which are incorporated herein by reference.

### FIELD

Embodiments described herein relate generally to an image processing apparatus.

### BACKGROUND

Conventionally, in breast cancer screening, a mammography image captured by a breast X-ray radiographic apparatus (hereinafter, referred to as a mammography apparatus) and an ultrasound image captured by an ultrasonic diagnostic apparatus are used. Specifically, in breast cancer screening, if a radiologist interprets a mammography image to find an area suspected of being a breast cancer (hereinafter, referred to as a region of interest (ROI)), the radiologist interprets an ultrasound image at a position substantially the same as that of the ROI. This makes it possible to carry out a diagnosis more accurately. In the conventional technology, however, it may possibly be difficult to interpret the ultrasound image including the ROI. A conventional example is described in Japanese Patent Application Laid-open No. 2011-110429.

### BRIEF DESCRIPTION OF THE DRAWINGS

FIG. 1 is a diagram of an example of a configuration of an image processing system according to a first embodiment;

FIG. 2 is a schematic of an example of a configuration of a mammography apparatus according to the first embodiment;

FIG. 3 is a schematic of an example of a configuration of an ultrasonic diagnostic apparatus according to the first embodiment;

FIG. 4 is a diagram of an example of a configuration of an image processing apparatus according to the first embodiment;

FIG. 5 is a view for explaining an example of processing performed by an identifying unit according to the first embodiment;

FIG. 6 is a schematic of a first display example of an ultrasound image according to the first embodiment;

FIG. 7 is a schematic of a second display example of the ultrasound image according to the first embodiment;

FIG. 8 is a schematic of a third display example of the ultrasound image according to the first embodiment;

FIG. 9 is a flowchart of a process performed by the image processing apparatus according to the first embodiment;

FIG. 10A is a view for explaining differences in a scanning process according to a second embodiment;

FIG. 10B is a view for explaining differences in the scanning process according to the second embodiment;

FIG. 10C is a view for explaining differences in the scanning process according to the second embodiment;

FIG. 11 is a diagram of an example of a configuration of an image processing apparatus according to the second embodiment;

FIG. 12 is a view schematically illustrating an example of processing performed by a rearranging unit according to the second embodiment;

FIG. 13 is a flowchart of a process performed by the image processing apparatus according to the second embodiment;

FIG. 14 is a schematic of a first display example of an ultrasound image according to a third embodiment; and

FIG. 15 is a schematic of a second display example of the ultrasound image according to the third embodiment.

### DETAILED DESCRIPTION

According to embodiment, an image processing apparatus comprising, a receiving unit, an identifying unit and a display control. The receiving unit that receives specification of a region of interest (ROI) included in a mammography image. The an identifying unit that identifies a medical image including a position substantially the same as a position of the ROI received by the receiving unit in a medical image group acquired from a subject for whom the mammography image is captured. The display control unit that performs control such that the medical image identified by the identifying unit is displayed on a predetermined display unit.

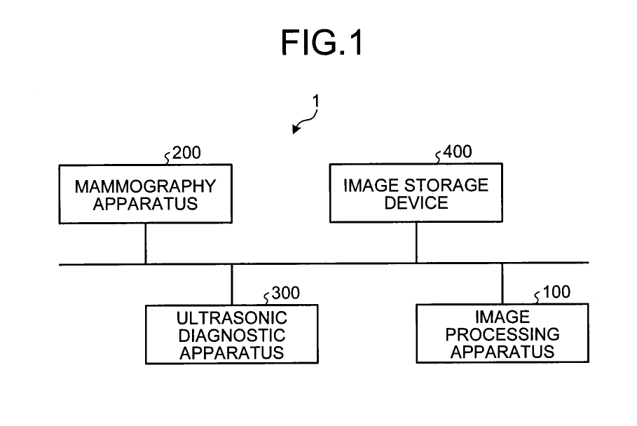

Exemplary embodiments of an image processing apparatus according to the present disclosure are described below in greater detail. In a first embodiment, an explanation will be made of an image processing system including the image processing apparatus according to the present disclosure. Furthermore, an explanation will be made of the case where an ultrasound image at a position substantially the same as that of a mammography image is identified. FIG. 1 is a diagram of an example of a configuration of the image processing system according to the first embodiment.

As illustrated in FIG. 1, an image processing system 1 according to the first embodiment includes an image processing apparatus 100, a mammography apparatus 200, an ultrasonic diagnostic apparatus 300, and an image storage device 400. The apparatuses illustrated in FIG. 1 are communicable with one another directly or indirectly via an in-hospital local area network (LAN) installed in a hospital, for example. If a picture archiving and communication system (PACS) is introduced into the image processing system 1, for example, the apparatuses transmit and receive medical images and the like to and from one another in accordance with the digital imaging and communications in medicine (DICOM).

In the image processing system 1, the mammography apparatus 200 and the ultrasonic diagnostic apparatus 300 acquire a mammography image and an ultrasound image, respectively, in response to an operation performed by respective technologists. The image processing apparatus 100 displays an image in response to an operation performed by a radiologist. Thus, the radiologist can perform breast cancer screening or the like by interpreting the mammography image or the ultrasound image.

The following describes the case where interpretation of an ultrasound image including a region of interest (ROI) is difficult to make in the conventional technology. In the conventional technology, for example, a technologist who acquires an ultrasound image performs scanning on the whole breast of a subject to be subjected to breast cancer screening. The technologist performs a saving operation at a desired timing for saving during the scanning. Thus, an ultrasound image is saved when the saving operation is performed. In other words, in the conventional technology, the ultrasound image thus saved depends on a skill of the technologist who acquires the ultrasound image. As a result, even if a radiologist interprets a mammography image to find a ROI and tries to interpret an ultrasound image, for example, an ultrasound image corresponding to the ROI may not possibly be saved. This makes it difficult to interpret the ultrasound image including the ROI. If all the ultrasound images obtained by the scanning are saved to address the problem described above, it takes time and effort to extract the ultrasound image including the ROI from all the ultrasound images. This increases a burden on the radiologist and makes it difficult to interpret the ultrasound image including the ROI.

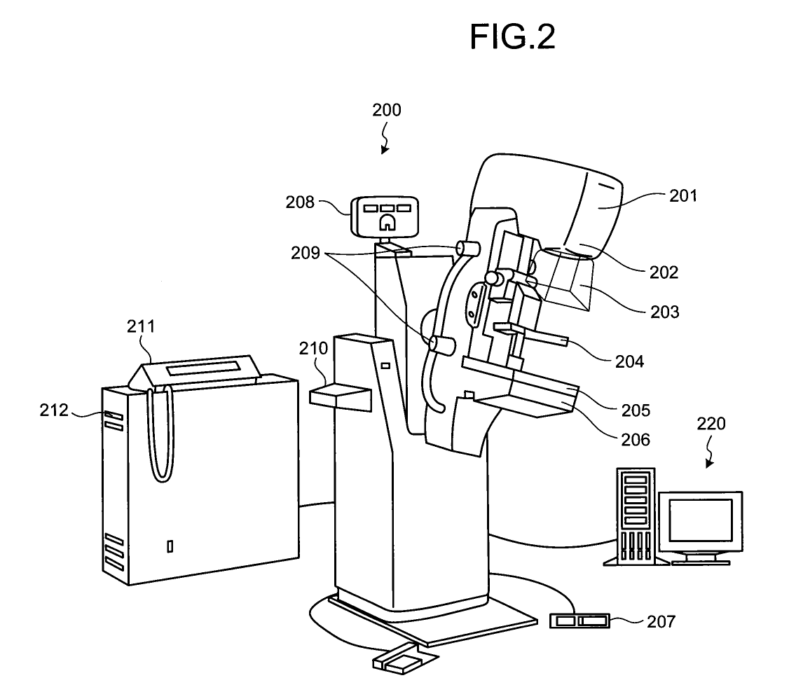

With a configuration described below in detail, the image processing system according to the first embodiment can facilitate interpretation of an ultrasound image including a ROI without increasing the burden on the radiologist. The following describes in detail each apparatus included in the image processing system according to the first embodiment. FIG. 2 is a schematic of an example of a configuration of the mammography apparatus 200 according to the first embodiment.

As illustrated in FIG. 2, the mammography apparatus 200 according to the first embodiment is formed of a radiographic table apparatus including an X-ray tube 201, a radiation quality control filter/radiation field limiting mask 202, a face guard 203, a breast pressing plate 204, a grid 205, a radiographic table 206, a compression foot pedal 207, an information display panel 208, a C-arm elevation and rotation fine-adjustment switch 209, a side panel 210, a radiography condition setting panel 211, and an X-ray high-voltage generator 212, and an image processing apparatus 220 connected to each other.

The X-ray tube 201 is a vacuum tube that generates X-rays. The radiation quality control filter/radiation field limiting mask 202 is a control member that controls the radiation quality of the X-rays generated by the X-ray tube 201 and limits the radiation field. The face guard 203 is a protective member that protects a subject while radiography is being performed. The breast pressing plate 204 is a pressing member that presses a breast of the subject while radiography is being performed.

The grid 205 is a member that eliminates scattered radiation and improves image contrast. The radiographic table 206 is a table including a flat panel detector (FPD) (an image detector) that detects X-rays passing through the breast. The compression foot pedal 207 is a pedal used to adjust the position of the breast pressing plate 204 in the vertical direction. The information display panel 208 is a panel that displays various types of information, such as pressure information.

The C-arm elevation and rotation fine-adjustment switch 209 is a switch used to lift up and down and rotate a C-arm formed of the X-ray tube 201, the radiographic table 206, and other components. The side panel 210 is an operation panel used to control each unit of the mammography apparatus 200. The radiography condition setting panel 211 is a panel used to set conditions for radiography. The X-ray high-voltage generator 212 is a device that supplies a voltage to the X-ray tube 201.

The image processing apparatus 220 is an apparatus that collectively controls the operation of the mammography apparatus 200 and performs image processing on a captured image captured by the mammography apparatus 200. If X-rays are generated by the X-ray tube 201, for example, the range of radiation of the X-rays is narrowed down by an X-ray movable diaphragm (not illustrated). A breast pressed between the breast pressing plate 204 and the radiographic table 206 is irradiated with the X-rays. The x-rays passing through the breast are detected by the FPD (not illustrated), converted into projection data, and transmitted to the image processing apparatus 220.

The image processing apparatus 220 receives the projection data transmitted from the radiographic table apparatus. The image processing apparatus 220 then generates a mammography image from the projection data thus received and transmits the mammography image thus generated to the image storage device 400. The image processing apparatus 220 includes an operating unit formed of a mouse, a keyboard, and other components and a monitor that displays various types of images generated based on projection data and displays a graphical user interface (GUI) used to receive various types of operations through the operating unit, for example.

With this configuration, the mammography apparatus 200 performs radiography at a position for “medio-lateral oblique (MLO) view” and a position for “cranio-caudal (CC) view” as radiography for breast cancer screening.

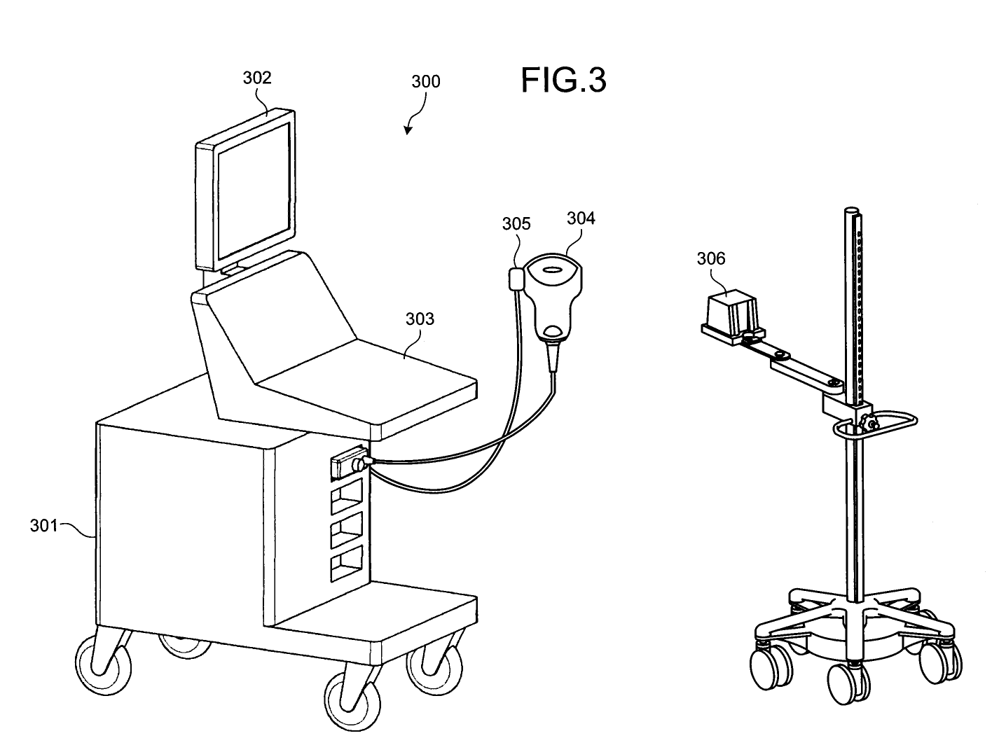

FIG. 3 is a schematic of an example of a configuration of the ultrasonic diagnostic apparatus 300 according to the first embodiment. As illustrated in FIG. 3, the ultrasonic diagnostic apparatus 300 according to the first embodiment includes an apparatus main body 301, a monitor 302, an operating unit 303, an ultrasonic probe 304, a position sensor 305, and a transmitter 306.

The apparatus main body 301 collectively controls the ultrasonic diagnostic apparatus 300. The apparatus main body 301, for example, performs various types of control related to generation of an ultrasound image. The monitor 302 displays a GUI through which an operator of the ultrasonic diagnostic apparatus 300 inputs various types of setting requests through the operating unit 303 and displays an ultrasound image generated by the apparatus main body 301.

The operating unit 303 includes a trackball, a switch, a button, a touch command screen, and the like. The operating unit 303 receives various types of setting requests from the operator of the ultrasonic diagnostic apparatus 300 and transmits the various types of setting requests thus received to the apparatus main body 301. The ultrasonic probe 304 transmits and receives ultrasonic waves. In the ultrasonic diagnostic apparatus 300 according to the first embodiment, the position sensor 305 is attached to the ultrasonic probe 304 as illustrated in FIG. 3. The position sensor 305 receives a signal transmitted from the transmitter 306, thereby detecting the position of the ultrasonic probe 304. Examples of the position sensor include a magnetic sensor, an infrared sensor, and an optical sensor.

The following describes the case where a magnetic sensor is used. In this case, the transmitter 306 is arranged at an arbitrary position and generates a magnetic field extending outward from the transmitter 306. The position sensor 305 attached to the surface of the ultrasonic probe 304 detects the three-dimensional magnetic field generated by the transmitter 306. The position sensor 305 then converts information of the magnetic field thus detected into a signal and outputs the signal to a signal processing device (not illustrated). Based on the signal received from the position sensor 305, the signal processing device derives the position (coordinates) and the direction of the position sensor 305 in a space with its origin at the transmitter 306. The signal processing device then outputs the information thus derived to the apparatus main body 301. The apparatus main body 301 adds information of the position and the direction of scanning to each ultrasound image obtained by the scanning with the ultrasonic probe 304. The apparatus main body 301 then transmits the ultrasound image to the image storage device 400.

To acquire an ultrasound image in breast cancer screening, for example, the apparatus main body 301 uses the position of the position sensor 305, the position of the transmitter 306, and the position of the xiphoid process of the subject, thereby deriving the position of the position sensor 305 with respect to the xiphoid process. Based on the position of the position sensor 305 with respect to the xiphoid process and physical data of the subject (e.g., height and weight), the apparatus main body 301 identifies the position of the ultrasonic probe 304 with respect the subject. The apparatus main body 301, for example, identifies which position in the left and right breasts of the subject is being scanned by the ultrasonic probe 304. The apparatus main body 301 transmits an ultrasound image to the image storage device 400 in association with the information of the position and the direction every time scanning is performed. In other words, the ultrasonic diagnostic apparatus 300 according to the first embodiment transmits all the ultrasound images obtained by scanning to the image storage device 400 in association with the information of the position and the direction.

The image storage device 400 is a database that stores therein medical images. Specifically, the image storage device 400 according to the first embodiment stores mammography images transmitted from the mammography apparatus 200, ultrasound images transmitted from the ultrasonic diagnostic apparatus 300, and the like in a storage unit and retains the images. In the first embodiment, the mammography images and the ultrasound images are each stored in the image storage device 400 in association with a subject ID, an examination ID, an apparatus ID, a series ID, and the like. Thus, the image processing apparatus 100 conducts a search using a subject ID, an examination ID, an apparatus ID, a series ID, and the like, thereby acquiring a desired mammography image and a desired ultrasound image from the image storage device 400. Furthermore, the ultrasound images are each associated with information of the position and the direction of scanning. Thus, the image processing apparatus 100 conducts a search using the information of the position and the direction, thereby acquiring a desired ultrasound image from the image storage device 400.

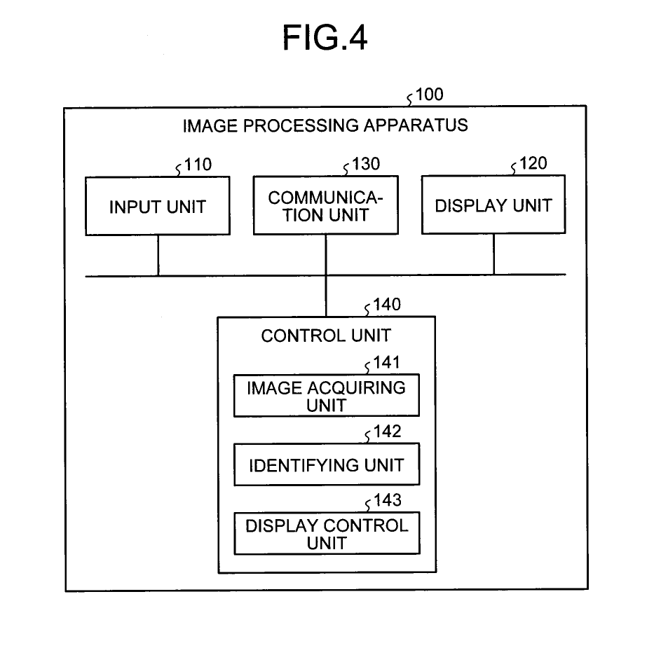

The image processing apparatus 100 according to the first embodiment will now be described. FIG. 4 is a diagram of an example of a configuration of the image processing apparatus 100 according to the first embodiment. As illustrated in FIG. 4, the image processing apparatus 100 includes an input unit 110, a display unit 120, a communication unit 130, and a control unit 140. The image processing apparatus 100, for example, is a workstation or an arbitrary personal computer. The image processing apparatus 100 is connected to the mammography apparatus 200, the ultrasonic diagnostic apparatus 300, the image storage device 400, and other apparatuses via a network.

The input unit 110 is formed of a mouse, a keyboard, a trackball, and other components. The input unit 110 receives input of various types of operations supplied to the image processing apparatus 100 from an operator (e.g., the radiologist). Specifically, the input unit 110 receives input of information used to acquire a mammography image and an ultrasound image from the image storage device 400. The input unit 110, for example, receives input for acquiring a mammography image obtained by capturing the breast of the subject subjected to breast cancer screening. Furthermore, the input unit 110 receives specification of a ROI (e.g., an area or a point of a focus indicating microcalcification or a specific lump) included in a mammography image.

The display unit 120 is a liquid crystal panel or the like serving as a monitor and displays various types of information. Specifically, the display unit 120 displays a GUI used to receive various types of operations from the operator and a mammography image, an ultrasound image, and the like acquired from the image storage device 400 by processing performed by the control unit 140, which will be described later. The images acquired by the control unit will be described later. The communication unit 130 is a network interface card (NIC) or the like and performs communications with the other apparatuses.

The control unit 140 is an electronic circuit, such as a central processing unit (CPU) and a micro processing unit (MPU), or an integrated circuit, such as an application specific integrated circuit (ASIC) and a field programmable gate array (FPGA), for example. The control unit 140 collectively controls the image processing apparatus 100.

As illustrated in FIG. 4, the control unit 140 includes an image acquiring unit 141, an identifying unit 142, and a display control unit 143, for example. The image acquiring unit 141 acquires a three-dimensional mammography image from the image storage device 400 via the communication unit 130. The image acquiring unit 141, for example, acquires a mammography image corresponding to information (e.g., a subject ID and an examination ID) input by the operator through the input unit 110 from the image storage device 400 via the communication unit 130. Furthermore, the image acquiring unit 141 acquires an ultrasound image identified by the identifying unit 142, which will be described later, from the image storage device 400. The mammography image and the ultrasound image acquired by the image acquiring unit 141 are stored in a memory area (not illustrated) included in the image acquiring unit 141 or a storage unit (not illustrated) included in the image processing apparatus 100. The storage unit is a hard disk or a semiconductor memory device, for example.

The identifying unit 142 identifies a medical image including a position substantially the same as that of a ROI received by the input unit 110 in a medical image group acquired from the subject for whom a mammography image is captured. The identifying unit 142, for example, identifies an ultrasound image obtained by scanning the position substantially the same as that of the ROI received by the input unit 110 in an ultrasound image group generated by scanning, with the ultrasonic probe, the subject for whom the mammography image is captured. For example, the display control unit 143, which will be described later, displays a mammography image acquired by the image acquiring unit 141 on the display unit 120. The input unit 110 receives specification of a ROI (e.g., an area or a point suspected of being a breast cancer) made by an observer (e.g., the radiologist) through the input unit 110 on the mammography image displayed on the display unit 120.

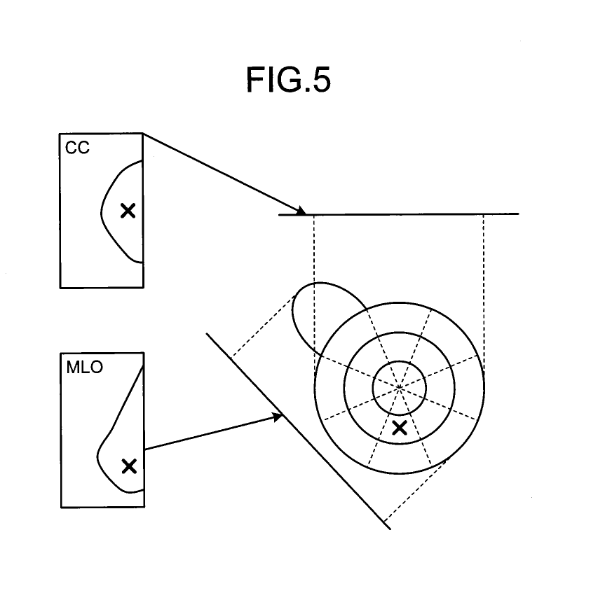

The identifying unit 142 identifies the position on the breast of the subject corresponding to the ROI received by the input unit 110. The identifying unit 142 then controls the image acquiring unit 141 so as to acquire an ultrasound image obtained by scanning a position substantially the same as the position thus identified from the image storage device 400. The following describes processing for identifying the position of the ROI performed by the identifying unit 142. FIG. 5 is a view for explaining an example of processing performed by the identifying unit 142 according to the first embodiment. FIG. 5 illustrates an example of processing for identifying the position in the breast corresponding to the ROI specified on a mammography image. FIG. 5 illustrates a breast captured in the CC view and the MLO view.

As illustrated in FIG. 5, for example, the identifying unit 142 models a breast from mammography images in the CC view and the MLO view. The identifying unit 142 divides the breast thus modeled into 24 sections (eight sections in the circumferential direction×three sections in the radial direction). The identifying unit 142 then identifies the position of the ROI in the breast based on the position of the ROI specified in the respective mammography images in the CC view and the MLO view. The number of sections of the breast may be arbitrarily set by the observer or a designer. The specification of the position of the ROI in the breast described above is given just as an example. The embodiment does not necessarily employ the method described above and may use other known technologies. The position of the ROI in the breast may be identified by deriving a distance from an anatomically characteristic portion, such as a line of skin, a nipple, and a chest wall, to the ROI thus specified.

The identifying unit 142 causes the image acquiring unit 141 to acquire an ultrasound image obtained by scanning the position substantially the same as that of the ROI thus specified. Specifically, the identifying unit 142 identifies an ultrasound image obtained by scanning the position substantially the same as that of the ROI thus specified based on the information of the scanning position and direction added to the ultrasound image. The identifying unit 142 then causes the image acquiring unit 141 to acquire the ultrasound image thus identified. The image acquiring unit 141 acquires the ultrasound image identified by the identifying unit 142 from the image storage device 400.

The information of the scanning position and direction of the ultrasound image referred to by the identifying unit 142 may be transmitted to the image processing apparatus 100 and stored in the storage unit, which is not illustrated, when the ultrasound image is stored in the image storage device 400. In this case, the identifying unit 142 accesses the storage unit, which is not illustrated, to refer to the information of the scanning position and direction of the ultrasound image. If the information of the scanning position and direction of the ultrasound image is not stored in the image processing apparatus 100, the identifying unit 142 may access the image storage device 400 via the communication unit 130 when identifying the ultrasound image. Thus, the identifying unit 142 may refer to the information of the scanning position and direction of the ultrasound image.

The display control unit 143 displays a mammography image acquired by the image acquiring unit 141 on the display unit 120. Specifically, the display control unit 143 displays a mammography image acquired by the image acquiring unit 141 and stored in the storage unit, which is not illustrated, on the display unit 120. The display control unit 143 displays a GUI used to specify a ROI in the mammography image on the display unit 120.

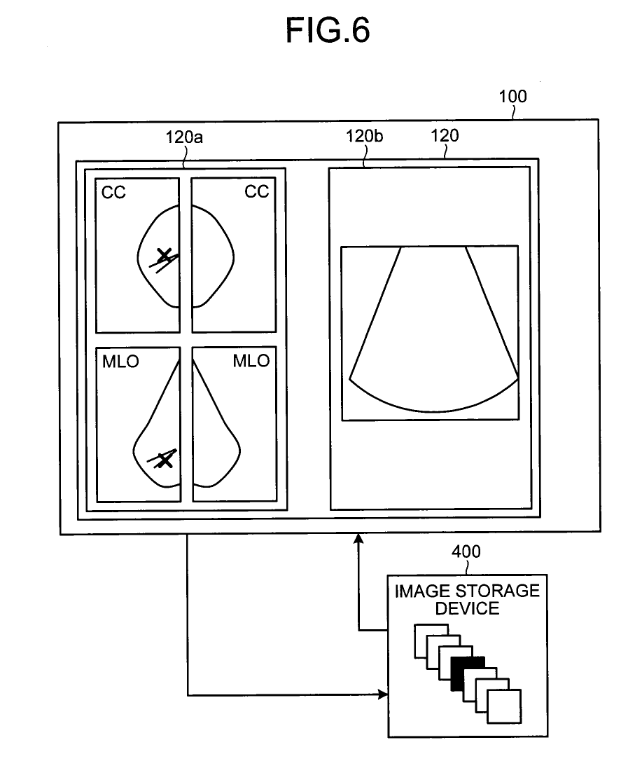

Furthermore, the display control unit 143 displays an ultrasound image acquired by the image acquiring unit 141 on the display unit 120. FIG. 6 is a schematic of a first display example of the ultrasound image according to the first embodiment. FIG. 6 illustrates display of a still image of the ultrasound image obtained by scanning the position substantially the same as that of the ROI. In the image processing apparatus 100 according to the first embodiment, the display unit 120 includes a mammography image display area 120a and an ultrasound image display area 120b as illustrated in FIG. 6. The display control unit 143 displays a mammography image in the CC view and a mammography image in the MLO view in the mammography image display area 120a.

If the observer (e.g., the radiologist) specifies a microcalcified area as the ROI, the identifying unit 142 identifies an ultrasound image obtained by scanning the position substantially the same as that of the ROI thus specified. The image acquiring unit 141 acquires the ultrasound image thus identified from the image storage device 400. The display control unit 143 displays the ultrasound image acquired by the image acquiring unit 141 in the ultrasound image display area 120b.

As described above, the image processing apparatus 100 according to the first embodiment acquires and displays the ultrasound image obtained by scanning the position substantially the same as that of the ROI specified in the mammography image from all the ultrasound images. This makes it possible to facilitate interpretation of the ultrasound image including the ROI.

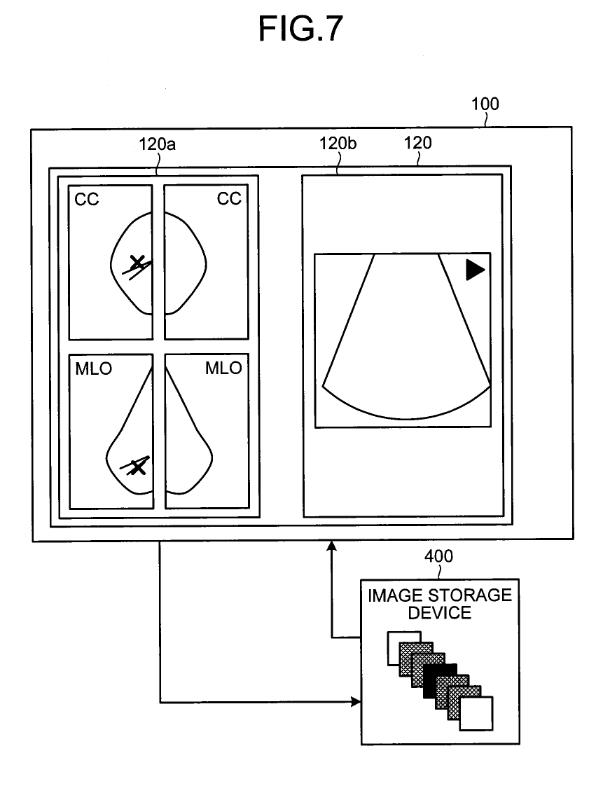

The image processing apparatus 100 according to the first embodiment can display the ultrasound image as a moving image besides as the still image described above. FIG. 7 is a schematic of a second display example of the ultrasound image according to the first embodiment. FIG. 7 illustrates display of a moving image of the ultrasound image obtained by scanning the position substantially the same as that of the ROI.

As illustrated in FIG. 7, for example, the image processing apparatus 100 according to the first embodiment acquires the ultrasound image (frame) obtained by scanning the position substantially the same as the position specified as the ROI and an arbitrary number of frames adjacent thereto as a moving image from the image storage device 400. The image processing apparatus 100 then displays the frames in the ultrasound image display area 120b of the display unit 120. In this case, the identifying unit 142 causes the image acquiring unit 141 to acquire the frame obtained by scanning the position substantially the same as that of the ROI specified in the mammography image and several frames before and after the frame. The display control unit 143 sequentially displays the frames acquired by the image acquiring unit 141 on the display unit 120. Thus, the display control unit 143 displays a moving image for the observer (e.g., the radiologist). This enables the radiologist to interpret the moving image near the ROI even if he/she does not know that the ultrasound image is stored as the moving image, for example.

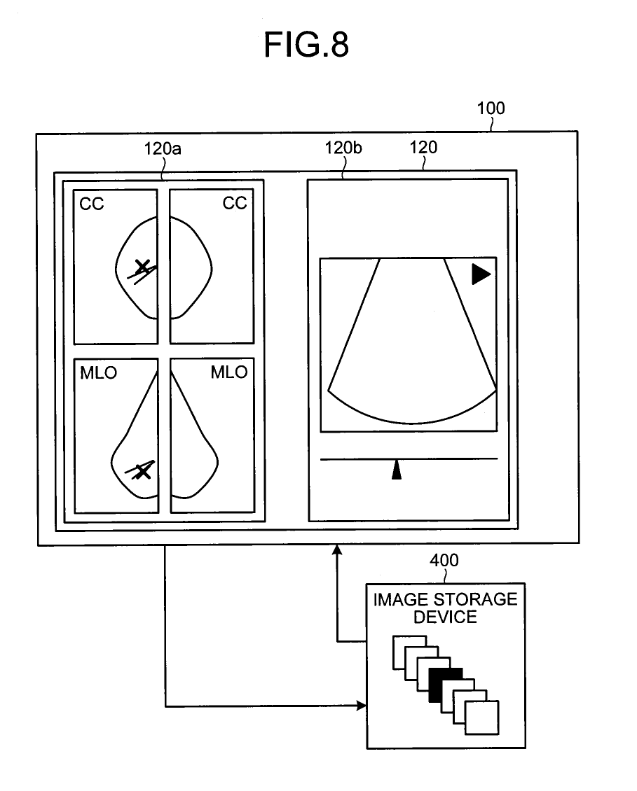

The image processing apparatus 100 according to the first embodiment can extract and display only the image including the ROI when the radiologist observes the moving image. FIG. 8 is a schematic of a third display example of the ultrasound image according to the first embodiment. As illustrated in FIG. 8, for example, the image processing apparatus 100 according to the first embodiment acquires the frame obtained by scanning the position substantially the same as the position specified as the ROI in the moving image from the image storage device 400. The image processing apparatus 100 then skips to the frame thus acquired and displays the frame in the ultrasound image display area 120b of the display unit 120 when the radiologist observes the moving image.

The three display formats described above can be arbitrarily set by the radiologist and other observers. Furthermore, the three display formats can be automatically switched depending on the state of acquisition of the ultrasound image thus stored (whether the ultrasound image is acquired as a moving image, for example).

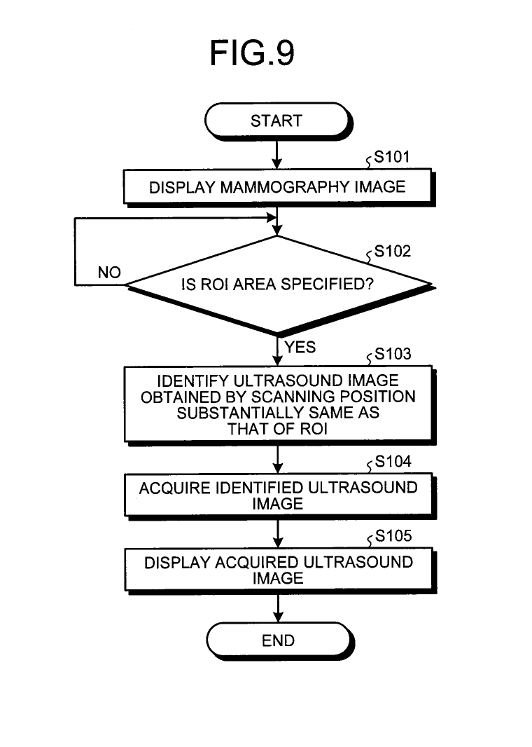

The following describes a process performed by the image processing apparatus 100 according to the first embodiment. FIG. 9 is a flowchart of a process performed by the image processing apparatus 100 according to the first embodiment. FIG. 9 illustrates processing performed after the mammography apparatus 200 and the ultrasonic diagnostic apparatus 300 acquire respective images and the images thus acquired are stored in the image storage device 400.

As illustrated in FIG. 9, in the image processing apparatus 100 according to the first embodiment, the image acquiring unit 141 acquires a mammography image from the image storage device 400 based on information (e.g., a subject ID and an examination ID) received by the input unit 110. The display control unit 143 displays the mammography image thus acquired on the display unit 120 (Step S101). If the mammography image is displayed, the identifying unit 142 determines whether a ROI is specified (Step S102).

If a ROI is specified (Yes at Step S102), the identifying unit 142 identifies an ultrasound image obtained by scanning a position substantially the same as that of the ROI in a breast (Step S103). The image acquiring unit 141 acquires the ultrasound image identified by the identifying unit 142 from the image storage device 400 (Step S104).

Subsequently, the display control unit 143 displays the ultrasound image acquired by the image acquiring unit 141 on the display unit 120 (Step S105). The image processing apparatus 100 according to the first embodiment waits for specification until a ROI is specified (No at Step S102).

As described above, according to the first embodiment, the input unit 110 receives specification of a ROI included in a mammography image. The identifying unit 142 identifies an ultrasound image obtained by scanning a position substantially the same as that of the ROI received by the input unit 110 in an ultrasound image group generated by scanning, with the ultrasonic probe, the subject for whom the mammography image is captured. The display control unit 143 performs control so as to display the ultrasound image identified by the identifying unit 142 on the display unit 120. Thus, the image processing apparatus 100 according to the first embodiment acquires and displays the ultrasound image obtained by scanning the position substantially the same as that of the ROI specified in the mammography image from all the ultrasound images. This makes it possible to facilitate interpretation of the ultrasound image including the ROI. As a result, the image processing apparatus 100 according to the first embodiment can reduce the burden on the radiologist and make the interpretation more efficient, thereby increase the accuracy of diagnosis.

According to the first embodiment, the identifying unit 142 identifies the position of the ROI in the breast of the subject based on the ROI specified in a mammography image in the CC view and a mammography image in the MLO view. The identifying unit 142 then identifies the ultrasound image obtained by scanning the position substantially the same as the position thus identified from the ultrasound image group to which positional information is added. Thus, the image processing apparatus 100 according to the first embodiment can identify the position using the images conventionally used for interpretation. This makes it possible to facilitate identifying the position precisely.

According to the first embodiment, the identifying unit 142 identifies the ultrasound image obtained by scanning the position substantially the same as that of the ROI or a plurality of ultrasound images including the ultrasound image and ultrasound images before and after the ultrasound image in chronological order in the ultrasound image group thus generated. The display control unit 143 displays the ultrasound image obtained by scanning the position substantially the same as that of the ROI or the ultrasound images including the ultrasound image and ultrasound images before and after the ultrasound image in chronological order identified by the identifying unit 142 on the display unit 120. Thus, the image processing apparatus 100 according to the first embodiment can display the ultrasound image in various display formats, thereby enabling accurate interpretation.

In the first embodiment, to display the ultrasound image as a moving image, the image processing apparatus 100 acquires a plurality of frames near the frame obtained by scanning the position substantially the same as that of the ROI, thereby displaying a moving image. An image processing apparatus 100a according to a second embodiment acquires a plurality of frames obtained by scanning a position substantially the same as that of a ROI and the vicinity thereof from all the frame data of a moving image.

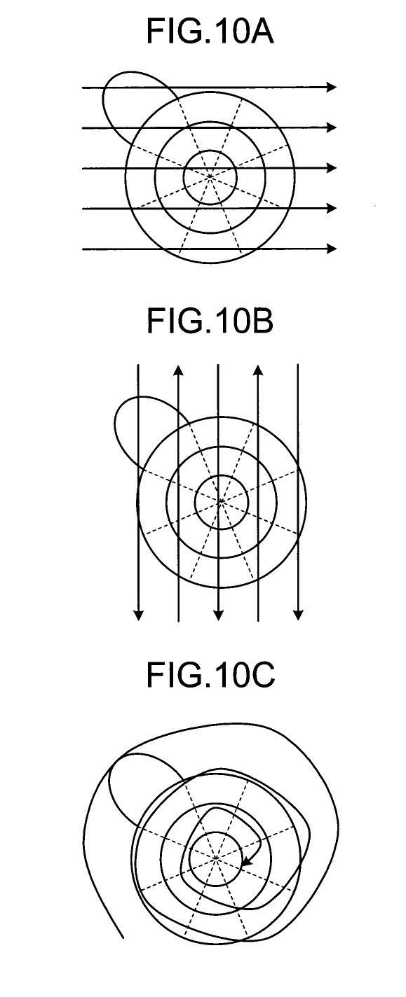

In scanning of a breast with an ultrasonic probe, for example, the process for scanning varies depending on technologists. FIGS. 10A to 100 are views for explaining differences in the scanning process according to the second embodiment. In FIGS. 10A to 10C, the direction in which the scanning is performed with respect to the breast is indicated by an arrow. As illustrated in FIG. 10A, for example, the scanning process may include scanning in one direction from left to right gradually from the upper portion to the lower portion of the breast in FIG. 10A. As illustrated in FIG. 10B, for example, the scanning process may include scanning in two directions from the upper portion to the lower portion of the breast or from the lower portion to the upper portion thereof in FIG. 10B. As illustrated in FIG. 100, for example, the scanning process may include scanning helically from the outside of the breast to the nipple in FIG. 100.

As described above, in scanning of the breast with the ultrasonic probe, the process for scanning varies depending on the technologists. As a result, in frames stored as a moving image, areas adjacent to one another in the breast are not necessarily stored as consecutive frames. The image processing apparatus 100a according to the second embodiment acquires frames of areas adjacent to one another in the breast and consecutively displays the frames thus acquired. Thus, the image processing apparatus 100a fully displays a frame of a position substantially the same as that of a ROI and frames of the vicinity thereof to the radiologist.

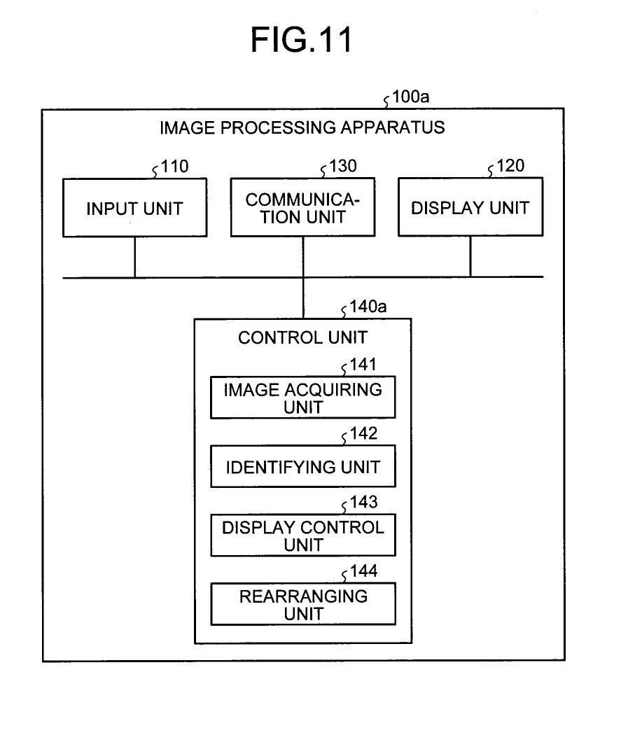

FIG. 11 is a diagram of an example of a configuration of an image processing apparatus 100a according to the second embodiment. In FIG. 11, the image processing apparatus 100a is different from the image processing apparatus 100 according to the first embodiment in that a control unit 140a includes a rearranging unit 144. In the description below, the rearranging unit 144 is mainly described.

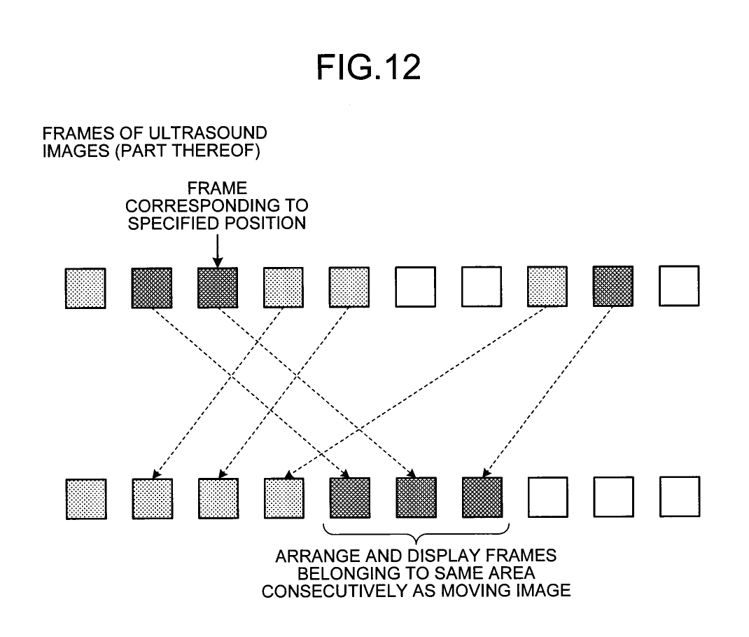

In a moving image of an ultrasound image stored in an image storage device 400, the rearranging unit 144 rearranges frames of the moving image of the ultrasound image such that frames whose scanning areas are adjacent to one another are consecutively arranged. Specifically, based on information of the scanning position and direction added to each frame, the rearranging unit 144 rearranges the frames such that frames whose scanning areas are adjacent to one another are consecutively arranged. FIG. 12 is a view schematically illustrating an example of processing performed by the rearranging unit 144 according to the second embodiment. FIG. 12 illustrates a part of frames of an ultrasound image of a certain subject stored in the image storage device 400. In FIG. 12, frames obtained by scanning areas adjacent to one another in a breast are indicated by similar density.

As illustrated in FIG. 12, for example, the rearranging unit 144 rearranges the frames of the ultrasound image stored in the image storage device 400 such that a frame obtained by scanning a position substantially the same as that of a ROI that is specified and frames obtained by scanning the vicinity thereof are consecutively arranged. Similarly, the rearranging unit 144 rearranges the frames such that frames obtained by scanning areas adjacent to one another in the breast are consecutively arranged. The rearranging unit 144 rearranges the frames based on the information of the scanning position and direction added to each frame. While the frames are rearranged after the frame obtained by scanning the position substantially the same as that of the ROI is identified in the example described above, the embodiment does not necessarily employ the process. The frames, for example, may be rearranged after the ultrasound image is stored in the image storage device 400 and before the frame obtained by scanning the position substantially the same as that of the ROI is identified.

An image acquiring unit 141 acquires the frame obtained by scanning the position substantially the same as that of the ROI and several frames before and after the frame from the frames rearranged by the rearranging unit 144. A display control unit 143 displays the frames acquired by the image acquiring unit 141 as a moving image on a display unit 120. This makes it possible to fully display the ultrasound images of the position substantially the same as that of the ROI and the vicinity thereof.

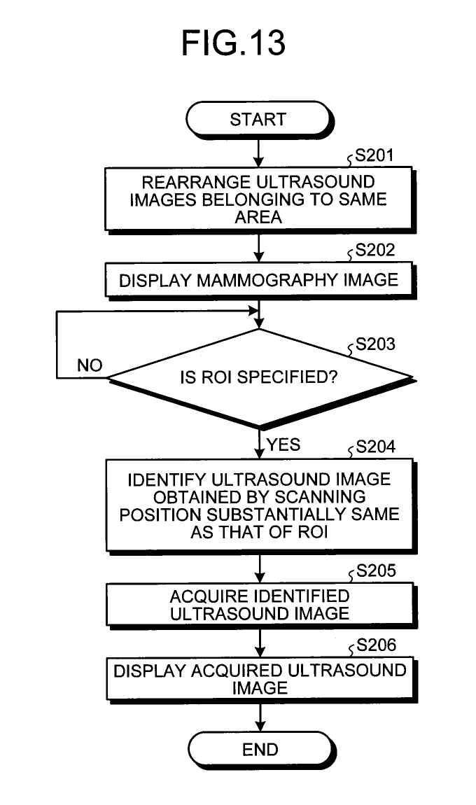

The following describes a process performed by the image processing apparatus 100a according to the second embodiment. FIG. 13 is a flowchart of a process performed by the image processing apparatus 100a according to the second embodiment. FIG. 13 illustrates processing performed after a mammography apparatus 200 and an ultrasonic diagnostic apparatus 300 acquire respective images and the images thus acquired are stored in the image storage device 400. Furthermore, FIG. 13 illustrates the case where rearrangement is performed before the frame obtained by scanning the position substantially the same as that of the ROI is identified.

As illustrated in FIG. 13, in the image processing apparatus 100a according to the second embodiment, if an ultrasound image is stored in the image storage device 400, the rearranging unit 144 rearranges the frames such that ultrasound images (frames) belonging to the same area in a breast are consecutively arranged (Step S201). Subsequently, the image acquiring unit 141 acquires a mammography image from the image storage device 400 based on information (e.g., a subject ID and an examination ID) received by an input unit 110. The display control unit 143 displays the mammography image thus acquired on the display unit 120 (Step S202). If the mammography image is displayed, an identifying unit 142 determines whether a ROI is specified (Step S203).

If a ROI is specified (Yes at Step S203), the identifying unit 142 identifies an ultrasound image obtained by scanning a position substantially the same as that of the ROI in the breast (Step S204). The image acquiring unit 141 acquires the ultrasound image identified by the identifying unit 142 from the image storage device 400 (Step S205).

Subsequently, the display control unit 143 displays the ultrasound image acquired by the image acquiring unit 141 on the display unit 120 (Step S206). The image processing apparatus 100a according to the second embodiment waits for specification until a ROI is specified (No at Step S203). To rearrange the frames after the frame obtained by scanning the position substantially the same as that of the ROI is identified, the processing at Step S201 is performed between Step S204 and Step S205 in FIG. 13.

As described above, according to the second embodiment, the rearranging unit 144 rearranges the ultrasound image group such that ultrasound images whose scanning areas are adjacent to one another are arranged consecutively in chronological order. Thus, the image processing apparatus 100a according to the second embodiment can fully display the ultrasound images of the position substantially the same as that of the ROI and the vicinity thereof.

While the first and the second embodiments have been described, the apparatus of the present application is applicable to various different embodiments besides the first and the second embodiments.

The first and the second embodiments use positional information acquired by the magnetic sensor, thereby adding information indicating which area of the subject is scanned to form the ultrasound image. The embodiments, however, do not necessarily employ the method described above. The embodiments, for example, may use an infrared sensor or an optical sensor, thereby adding information indicating which area of the subject is scanned to form the ultrasound image.

Besides the positional sensor described above, the embodiments may use an automated breast ultrasound system (ABUS), thereby adding positional information to the ultrasound image, for example. The ABUS is an automatic ultrasonic apparatus for a breast. The ABUS mechanically performs scanning with an ultrasonic probe and stores therein ultrasound images of the whole breast. It is also known that the ABUS has a 3D reconstruction function.

In the ABUS, if a box-shaped device having a built-in ultrasonic probe is set above the breast of the subject, for example, the ultrasonic probe automatically moves in a parallel direction to scan the whole breast. The ABUS acquires volume data (three-dimensional data) obtained by scanning the whole breast. Because the ultrasonic probe scans the whole breast while moving automatically at a constant speed in the ABUS, it is possible to identify which area of the breast is scanned to form the ultrasound image acquired by the ABUS. The ABUS is applied to an ultrasonic diagnostic apparatus 300 according to a third embodiment. Every time an ultrasound image is acquired, the ultrasonic diagnostic apparatus 300 adds positional information to each frame and transmits the frame to an image storage device 400.

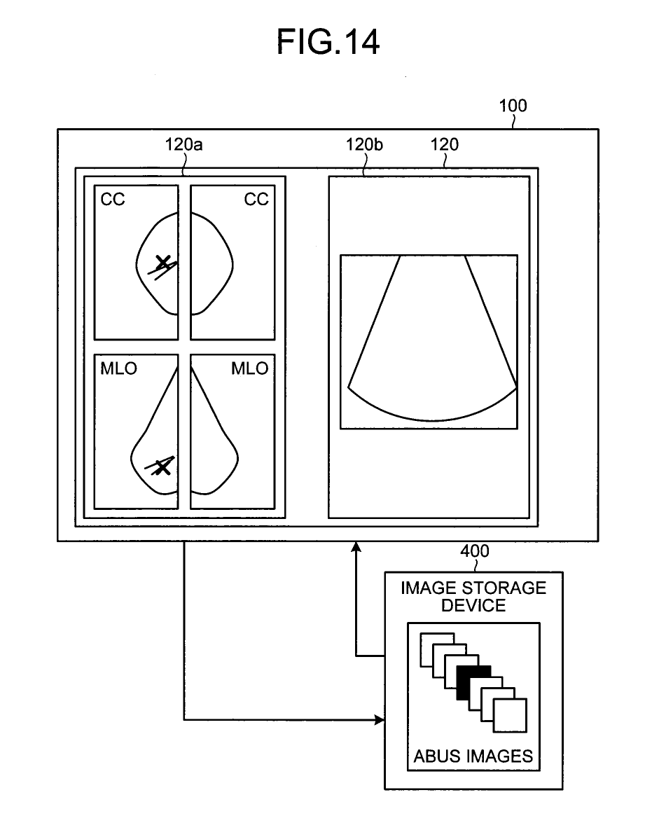

FIG. 14 is a schematic of a first display example of an ultrasound image according to the third embodiment. FIG. 14 illustrates display of a still image of the ultrasound image obtained by scanning a position substantially the same as that of a ROI. In an image processing apparatus 100 according to the third embodiment, as illustrated in FIG. 14, a display control unit 143 displays a mammography image in the CC view and a mammography image in the MLO view in a mammography image display area 120a.

If the radiologist specifies a ROI, an identifying unit 142 identifies an ultrasound image obtained by scanning the position substantially the same as that of the ROI thus specified. An image acquiring unit 141 acquires, from the image storage device 400, the ultrasound image identified by the identifying unit 142 from ABUS images. The display control unit 143 displays the ultrasound image acquired by the image acquiring unit 141 in an ultrasound image display area 120b.

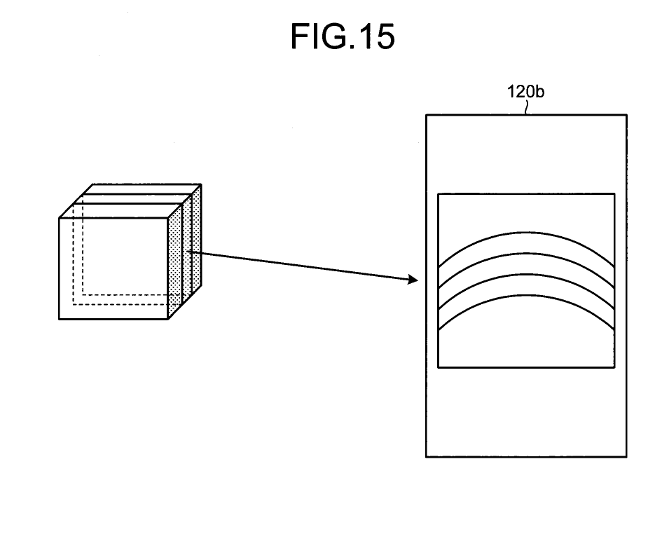

Because the ABUS has a 3D reconstruction function as described above, the image processing apparatus 100 according to the third embodiment can display a 2D image obtained by projecting a certain area of volume data. FIG. 15 is a schematic of a second display example of the ultrasound image according to the third embodiment. As illustrated in FIG. 15, for example, the image processing apparatus 100 according to the third embodiment displays, in the ultrasound image display area, a two-dimensional ultrasound image obtained by projecting an area including the ROI in the volume data. Thus, the image processing apparatus 100 according to the third embodiment can display the state of the ROI in the breast more clearly, thereby further increasing the diagnostic accuracy.

In the first embodiment, the explanation has been made of the case where the image processing apparatus 100 operates in a stand-alone manner. The embodiment, however, does not necessarily employ the configuration. The image processing apparatus may be integrated into the mammography apparatus or the ultrasonic diagnostic apparatus, for example.

In the first embodiment, the explanation has been made of the case where the image storage device 400 is connected to the network, and mammography images and ultrasound images are stored in the image storage device 400. The embodiment, however, does not necessarily employ the configuration. The mammography images and the ultrasound images may be stored in any one of the image processing apparatus 100, the mammography apparatus 200, and the ultrasonic diagnostic apparatus 300, for example.

In the embodiments, the explanation has been made of the case where the image processing apparatus 100 identifies an ultrasound image obtained by scanning a position substantially the same as that of a ROI and acquires only images related to the ultrasound image thus identified from the image storage device 400. The embodiments, however, do not necessarily employ the method. The image processing apparatus 100 may acquire all the ultrasound images corresponding to a subject ID and an examination ID that are specified from the image storage device 400 and store all the ultrasound images in the storage unit of the image processing apparatus 100, for example. The image processing apparatus 100 may then read images related to the ultrasound image thus identified from the storage unit and display the images on the display unit.

In the embodiments, the explanation has been made of the case where an ultrasound image of a position substantially the same as that of a ROI specified in a mammography image is identified and displayed. The embodiments, however, do not necessarily employ the method. An image of the position substantially the same as that of the ROI specified in the mammography image may be identified and displayed from an MR image acquired by a magnetic resonance imaging (MRI) apparatus and a CT image acquired by an X-ray computed tomography (CT) apparatus, for example.

In this case, the identifying unit 142 identifies the image of the position substantially the same as that of the ROI specified in the mammography image based on an anatomically characteristic portion, such as a line of skin and a xiphoid process, in the MR image or the CT image. The embodiments are given just as an example. The embodiments do not necessarily employ the method described above and may use other known technologies.

The image processing apparatus according to any one of the embodiments can facilitate interpretation of an ultrasound image including a ROI.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising:
a receiving unit that receives specification of a region of interest (ROI) included in a mammography image;
an identifying unit that identifies a medical image including a position substantially the same as a position of the ROI received by the receiving unit in a medical image group acquired from a subject for whom the mammography image is captured; and
a display control unit that performs control such that the medical image identified by the identifying unit is displayed on a predetermined display unit.

2. The image processing apparatus according to claim 1, wherein the identifying unit identifies the position of the ROI in a breast of the subject based on the region of interest specified in a mammography image in a cranio-caudal (CC) view and a mammography image in a medio-lateral oblique (MLO) view and identifies the medical image including the position substantially the same as the position thus identified in the medical image group.

3. The image processing apparatus according to claim 1, wherein
the identifying unit identifies an ultrasound image obtained by scanning the position substantially the same as the position of the ROI or a plurality of ultrasound images including the ultrasound image and ultrasound images before and after the ultrasound image in chronological order in an ultrasound image group generated by scanning, with an ultrasonic probe, the subject for whom the mammography image is captured, and
the display control unit displays the ultrasound image obtained by scanning the position substantially the same as the position of the ROI or the ultrasound images including the ultrasound image and ultrasound images before and after the ultrasound image in chronological order identified by the identifying unit on the predetermined display unit.

4. The image processing apparatus according to claim 1, further comprising a rearranging unit that rearranges an ultrasound image group generated by scanning, with an ultrasonic probe, the subject for whom the mammography image is captured such that ultrasound images whose scanning areas are adjacent to one another are arranged consecutively in chronological order.

5. The image processing apparatus according to claim 1, wherein, when an ultrasound image is generated by scanning, with an ultrasonic probe, the subject for whom the mammography image is captured, positional information acquired by a position sensor attached to the ultrasonic probe or an automated breast ultrasound system (ABUS) is added to the ultrasound image.

6. The image processing apparatus according to claim 5, wherein, when the positional information is acquired by the ABUS, the display control unit displays a two-dimensional image obtained by projecting a certain area in volume data acquired by the ABUS on the predetermined display unit.
